(19) United States
(12) Patent Application Publication
(10) Pub. No.: US 2018/0133500 A1
(43) Pub. Date: May 17, 2018

(54) Selective Cell Destruction through Electromagnetic Resonance

(71) Applicant: The Charles Stark Draper Laboratory, Inc., Cambridge, MA (US)

(72) Inventor: Andrew Cunningham, Somerville, MA (US)

(21) Appl. No.: 15/812,332

(22) Filed: Nov. 14, 2017

Related U.S. Application Data
(60) Provisional application No. 62/421,512, filed on Nov. 14, 2016.

(51) Int. Cl. A61N 5/02; C12N 13/00; A61N 5/06

(52) U.S. Cl. CPC A61N 5/025; A61N 5/0625; C12N 13/00

(57) ABSTRACT

Methods and devices for selectively destroying a cell or population of cells in cell cultures, fluids, blood or in the tissue or organs of a subject using electromagnetic resonance by targeting one, or more, specific protein(s) and/or nucleic acid(s) within the cell or cell population are described.

### RELATED APPLICATIONS

This application claims the benefit under 35 USC 119(e) of U.S. Provisional Application No. 62/421,512, filed on Nov. 14, 2016, which is incorporated herein by reference in its entirety.

### BACKGROUND OF THE INVENTION

In the fields of microbiology and medicine, a persistent challenge arises when attempting to destroy only one selected cell type while leaving all others intact. This is commonly done using chemical and biological agents which are developed to target known proteins, nucleic acids or other key molecules in e.g., metabolic pathways the cells. Such agents are tailored specifically to the target cell type.

On a microscopic level, for example, when dealing with a cell culture, isolating and destroying a specific cell type in that culture often requires the addition of an external chemical or biological agent, which in turn may disrupt the culture conditions affecting all growing cells and additionally requires time to propagate through the culture before acting on targeted cells.

Similarly, on a macroscopic level, infections in the human body, such as caused by bacteria and viruses, are difficult to target. Agents such as antibiotics and antiviral drugs created to target specific bacteria and viruses are often short-acting, or largely ineffective against many of the targeted harmful bacteria and viruses.

Also on a macroscopic level, cancer chemotherapy is an example of selective destruction of specific cells in a subject. Cancer, and the uncontrolled reproduction of cancer cells in a subject, continues to kill almost 600,000 patients in the US per year despite advances in chemical chemotherapy and localized radiation therapy [2016 Report on Cancer Status, National Cancer Institute].

In addition to chemical and biological means of targeting cells, in particular cancer cells, there are also available techniques which use high energy electrons (electron beam therapy) and high energy electromagnetic (EM) radiation (e.g., X-rays). In each case, distinguishing between target and off-target tissue is done only through physical localization and leads to severe off-target effects.

Therefore there still exists a great need for highly specific means to selectively and specifically target cells both in vitro and in vivo for destruction and elimination.

### SUMMARY OF THE INVENTION

As described herein, the present invention comprises methods and devices to use highly tuned electromagnetic radiation to specifically target cells of interest for destruction. Each cell is made up of many types of molecules, from unique and complex DNA molecules to millions of variations of proteins used for various functions throughout the cell. Many of these molecules have unique natural resonance energy states which enable them to be excited by infrared, terahertz or microwave electromagnetic radiation at particular frequencies corresponding to those energy states. Typically, higher energy infrared frequencies will excite chemical bonds between atoms (vibrational excitation) and terahertz and microwave frequencies will excite rotational states of molecules. Terahertz region radiation will also cause excitation from higher vibrational states to even higher vibrational states as the energy gap between states diminish. Such unique resonance properties can be identified (e.g., determined using methods known to those of skill in the art) and can be exploited (e.g., taken advantage of, or utilized, or made use of) resulting in methods to destroy a cell of interest or population of cells of interest using electromagnetic resonance by targeting one, or more, specific molecules, protein(s) and/or nucleic acid(s) within the cell or specific cells within a mixed cell population. The natural resonance of the molecules of interest (i.e., proteins or nucleic acids within target cells) will not change, and no external molecules or agents will be added, but the resonance properties can be utilized to excite/heat/denature the proteins and/or nucleic acid(s) within the targeted cell. Such methods and devices are described herein.

In particular, the methods comprise the steps of identifying the electromagnetic resonance properties of one, or more, target proteins, nucleic acids, molecules or molecular structures within the cell(s), for example for a particular protein type, or tuned specifically for a subject's cells. Once the electromagnetic resonance has been identified, an external energy source is provided. Such source can be of matching resonant frequency and capable of generating sufficient energy within the cell's, target protein/nucleic acid/molecule by exploiting the electromagnetic resonance properties previously identified and increase energy/heat within the target cell(s). The cell(s) are exposed to the energy source in a manner and for a time sufficient wherein exposure to the energy source results in increased energy/heat within the cell(s) and further results in the selective death of cell(s) containing, or comprising, the targeted protein/nucleic acid or molecule.

As a result of exposing the cells to the energy source, excitation of resonating proteins/nucleic acids/molecules occurs at a greater magnitude than the surrounding molecules and can increase the friction of intermolecular bonds of the target protein and/or nucleic acid resulting in increased heat within the cell. Alternatively, the exploitation of electromagnetic resonance properties within the proteins/nucleic acids/molecules increases the energy level within the molecular structure and totally or partially denatures the targeted protein and/or nucleic acid, resulting in the selective destruction of the targeted cell. (See FIGS. 1 and 2).

In particular, in one embodiment of the present invention, the one, or more targeted protein(s) and/or nucleic acid(s) and/or molecule(s) and/or cellular or molecular structure(s) are specific for certain types of cells such as cancer cells, bacterial cells or viruses. The use of the methods described herein results in the selective destruction of such cells, bacteria and viruses without significant harm to normal, non-malignant cells, or cells that are not infected with bacteria or virus, or the bacteria or a virus itself. Other cells that can be targeted for elimination using the methods described herein are, for example, cells with genetic defects resulting from abnormal/atypical nucleic acid sequences. Such atypical nucleic acid, such as a DNA or RNA, or other nucleic acid structure, can be targeted using methods described herein.

In another embodiment of the present invention, the methods described herein can be performed in vitro wherein the cells are maintained under cell culture conditions as known to those of skill in the art. Cell cultures typically contain a mixed population of cell types where, for example, only one cell population is desirable for continued culture. The methods described herein can be used to essentially eliminate completely, or significantly decrease, the contaminating cell types by targeting protein(s) and/or nucleic acid(s) and/or molecules that are specific for the contaminating cell types. (See FIG. 3).

Still another embodiment of the present invention is an ex vivo method of selectively destroying a cell, or population of cells, in a bodily fluid obtained from the subject, e.g., the blood of a subject, using electromagnetic resonance by targeting one, or more, specific protein(s) and/or nucleic acid(s) within the cell or a target cell within a mixed cell population. The subject can be a mammal, bird or reptile, and in particular the subject is a human.

The steps of the ex vivo methods comprise the steps of identifying the electromagnetic resonance properties of the target protein and/or nucleic acid within the cells; providing an external energy source capable of generating sufficient energy within the cells to exploit the electromagnetic resonance properties identified and increase the energy/heat within the cell(s); transporting the fluid/blood from the subject through a sterile device and exposing/irradiating the fluid/blood to the energy source provided for a time sufficient to treat the fluid/blood and increase energy/heat within the target cell(s) of the fluid, wherein the increased energy/heat within the cell(s) results in the selective death of cells containing the targeted protein/and or nucleic acid; and returning/transporting the treated fluid/blood back into the subject. (See FIGS. 4 and 5). Optionally the treated fluid/blood is transported through a filter device to eliminate the dead cells or other debris from the treated fluid/blood before returning/transporting the treated fluid/blood back into the subject. Such an ex vivo treatment is especially suitable as a cancer therapy.

Another embodiment of the present invention is a method of selectively destroying cells or a population of cells in the blood, tissue or organ of a subject (mammal, bird reptile or human) using electromagnetic resonance by targeting one, or more, specific protein(s) and/or nucleic acid(s) within the cells (see FIG. 6). Such methods can comprise the steps of: identifying the electromagnetic resonance properties of the target protein and/or nucleic acid within the cells; providing an external energy source capable of generating sufficient energy within the cells to modify the electromagnetic resonance properties as previously identified; and supporting and positioning the subject's body in proximity to the energy source and exposing the subject to the energy source in a manner and for a time sufficient for the increase of energy/heat within the target cell(s), wherein the increased energy/heat within the cell(s) results in the selective death of cells containing the targeted protein/and or nucleic acid, thus treating the subject. The irradiation can be applied to the whole body of the subject, or to localized areas.

Alternatively, if full body propagation cannot be obtained at the desired frequency and power/intensity for a blood-born target cell, the IR or other excitation could be applied in close proximity to a blood vessel as the targeted cells flow through the vessel and, thus, the cells would be destroyed without removing blood from the subject, effectively filtering the blood “in vivo” and, thus, treating the subject.

Other embodiments of the present invention are systems (especially comprising microfluidic devices) suitable for carrying out any of the above-described methods. In particular, the system comprises a device providing an external energy source capable of generating sufficient energy within the cells or population of cells to exploit the electromagnetic resonance properties of molecules exposed to the energy source. For example, one skilled in the art can determine the tight narrow electromagnetic radiation bandwidth whose frequency matches energy gaps of selected molecules and does not match energy gaps of non-selected molecules. Such a device can be, for example, a tunable near infrared laser device as described herein.

The above and other features of the invention including various novel details of construction and combinations of parts, and other advantages, will now be more particularly described with reference to the accompanying drawings and pointed out in the claims. It will be understood that the particular methods and devices embodying the invention are shown by way of illustration and not as a limitation of the invention. The principles and features of this invention may be employed in various and numerous embodiments without departing from the scope of the invention.

### BRIEF DESCRIPTION OF THE DRAWINGS

FIG. 1 is an outline describing the critical aspects and features of the invention.

FIG. 2 is a schematic drawing showing the results of heating proteins within a cell.

FIG. 3 is a schematic drawing depicting the method and device/system suitable for treating cell cultures to eliminate contaminating cells.

FIG. 4 is a drawing depicting the method and device/system suitable for the ex vivo treatment of blood from a subject by the present invention.

FIG. 5 is a photograph of a microfluidic device adaptable for use in the methods and system of the present invention.

FIG. 6 is a drawing depicting the method and device/system for whole body treatment using the methods of the present invention.

FIG. 7 is a precise schematic (implementation level embodiment) showing the laser pump source, optical fiber guide, tunable laser and the vial containing infected and healthy cells.

FIG. 8 details the sequential steps undertaken to find resonant frequencies and bandwidths using a COTS (Commercial Off The Shelf) laser system.

### DETAILED DESCRIPTION OF THE PREFERRED EMBODIMENTS

As described throughout this disclosure, photons, electromagnetic waves, electromagnetic radiation, and light will denote the same physical entity. X-rays are high energy (i.e., high frequency, short wavelength) photons and terahertz region has low energy photons (low frequency and long wavelength) in the far infrared region. More precisely, the infrared (IR) region is commonly defined from 700 nm to 1 mm, or frequencies of 430 THz to 300 GHz. (1 nm=10⁻⁹ m, 1 THz=10¹² cycles/sec and 1 GHz=10⁹ cycles/sec.) The terahertz region of electromagnetic spectrum (also denoted by THz) refers to the low frequency end of the IR region, whereas the near infrared (NIR) refers to the high frequency end of IR. The methods of this invention have applications for even lower frequencies, down to 10⁴ Hertz.

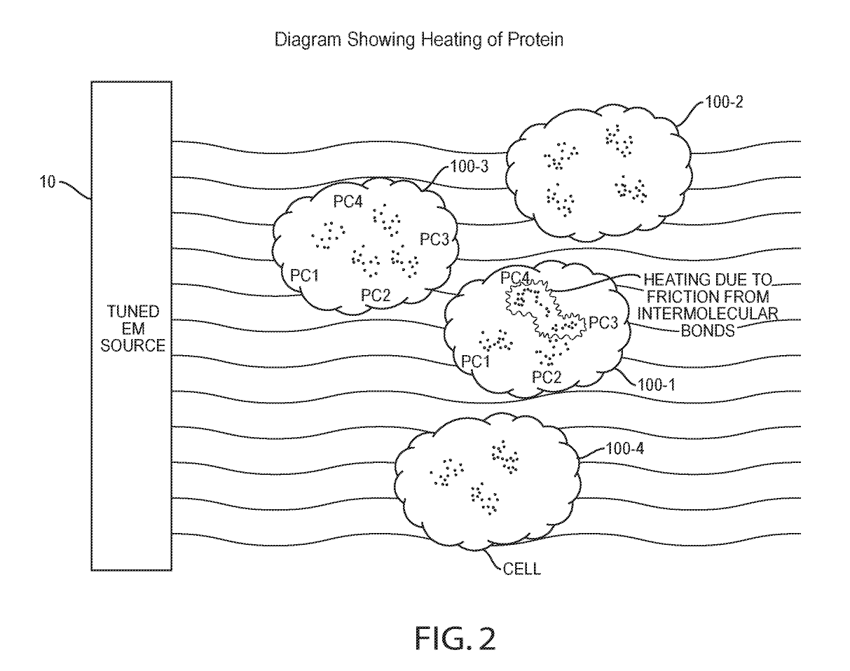

With advancements in precision electronics, fixed frequency resonators, and resonance prediction modeling in the last decade, it is reasonable to believe that it is possible to exploit the natural resonance properties of simple proteins or nucleic acids that occur naturally within a cell to add energy to the protein/nucleic acid molecule (and thus to the cell by conduction). Each protein or nucleic acid molecule has a single natural resonant frequency and multiple harmonic frequencies at which it will resonate when excited by a frequency-precise external energy source. Many cells have unique proteins or nucleic acids that can be identified as specific targets for the selected cell. Such proteins and/or nucleic acids are known to those of skill in the art, and more than one protein/nucleic acid can be identified for each cell type to increase the specificity of targeting the selected cell. When excited, energy from the external energy source will be captured in the protein or nucleic acid molecule, which can heat the cell and/or potentially denature the protein or nucleic acid. Adding energy in this way to a cell in a culture or a human body fluid would rapidly kill those specific cells, while causing limited harm to surrounding cells (see FIG. 2).

As described herein, the concept of intentionally using resonance as a destructive agent on specific intra- and extracellular proteins and intracellular nucleic acid molecules is novel. However, there is precedence to show that this type of targeted energy absorption (specifically terahertz radiation) is plausible for DNA and proteins. For example, during THz spectrum testing at Los Alamos National Laboratories, it was found that their particular THz field had a destructive force on human DNA in the skin, which proved to be a hazard for the operators. (DNA Breathing Dynamics in the Presence of a Terahertz Field, Los Alamos National Laboratory/Harvard Medical School; Vibrational Resonances in Biological Systems at Microwave Frequencies, Biophysical Journal 2002).

Targeting specific proteins and nucleic acids such as DNA, and other types of molecules requires identifying the exact resonance(s) of the target molecule. This is an advancing field of study with non-experimental prediction solutions emerging that have the capability of predicting protein electromagnetic resonances varying widely across the THz, GHz, MHz and kHz bands. (See “Is it possible to predict electromagnetic resonances in proteins, DNA and RNA?” EP Nonlinear Biomedical Physics, 2015). Some tools are also becoming available that can assist in preliminary studies, including an open system for defining dipole moments of arbitrary proteins. (See “A server and database for dipole moments of proteins”, Nucleic Acids Research, 2007). Therefore it is reasonable to believe that the modeling and quantitative estimation of specific protein/nucleic acid resonant frequencies (and harmonics) can be determined by one of skill in the art and applied to the methods and systems described herein.

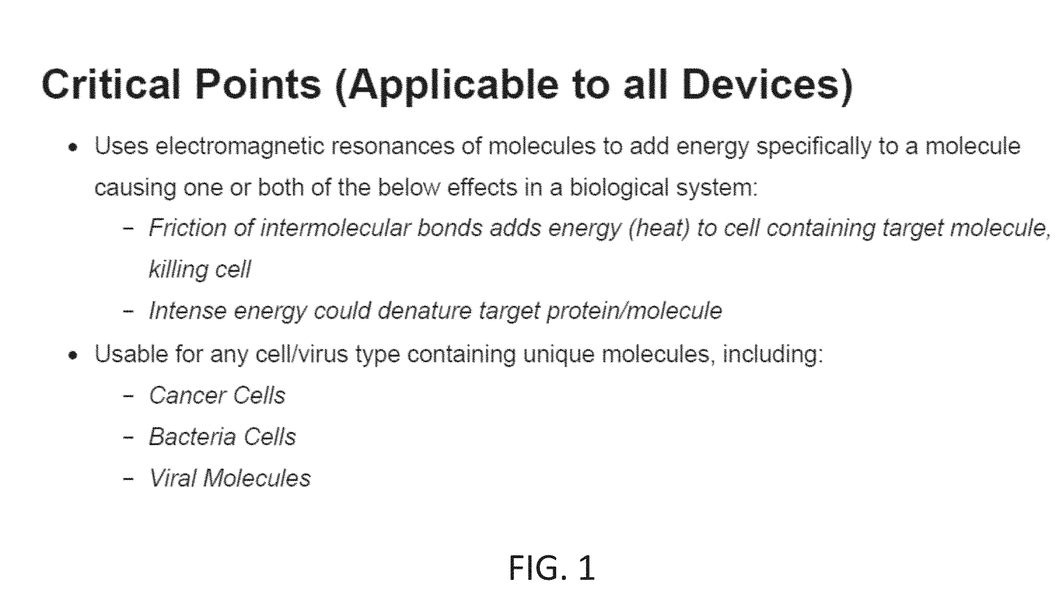

FIG. 1 is an outline of critical aspects of the invention applicable to all potential systems/devices. The invention uses electromagnetic radiation and more specifically radio frequency to near IR radiation, corresponding to resonance energy gaps of molecules to excite infected cells. Similar to how a microwave oven heats water and cooks food, the intra and intermolecular excitation of proteins/nucleic acids will heat a cell and lead to rupture/lysis (for example by intracellular protein denaturation), thus destroying the cell. As described herein, such methods can be used to kill cancerous cells, infected cells and bacteria and viruses.

FIG. 2 shows the process and result of applying the steps of this invention in a generic setting. As an example, four cells 100-1, 100-2, 100-3 and 100-4 are shown exposed to an electromagnetic terahertz radiation source 10. Each cell is shown to have four (an arbitrarily chosen number) protein clusters PC1, PC2, PC3 and PC4. The cell number 100-1 is presumed cancerous. The frequency (in a highly tuned narrow band) of the source is chosen to match the energy level gaps of infected protein clusters, i.e., it is in resonance with the said energy level gaps. However, the frequency does not match the said gaps of healthy protein clusters. After sufficiently long exposure to radiation, the cancerous cell 1001 shows the effect of terahertz irradiation. The cancerous cell's damaged protein clusters PC3 and PC4 absorb terahertz radiation and finally, overheat, resulting in the destruction of the cell. However, the impinging radiation does not affect the healthy protein clusters as it is not in resonance with energy levels of the healthy proteins.

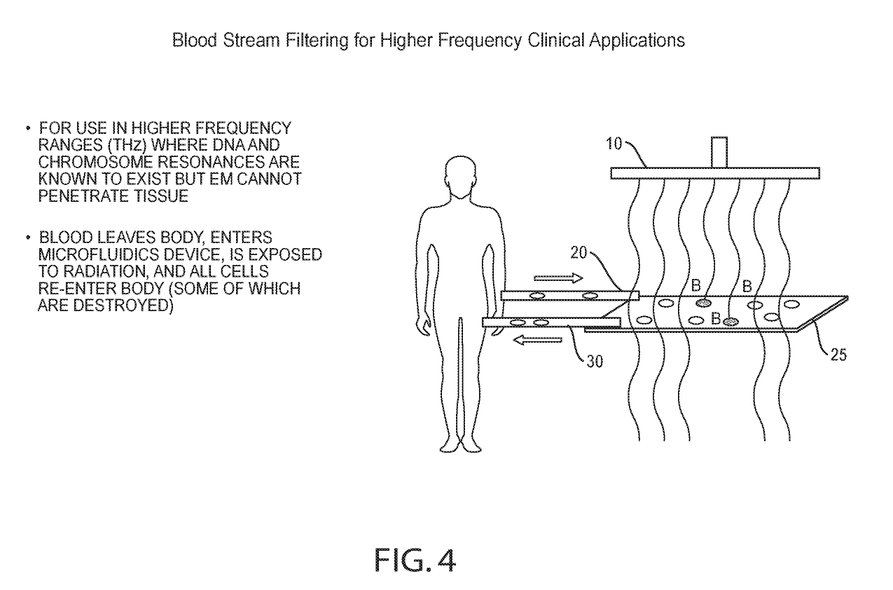

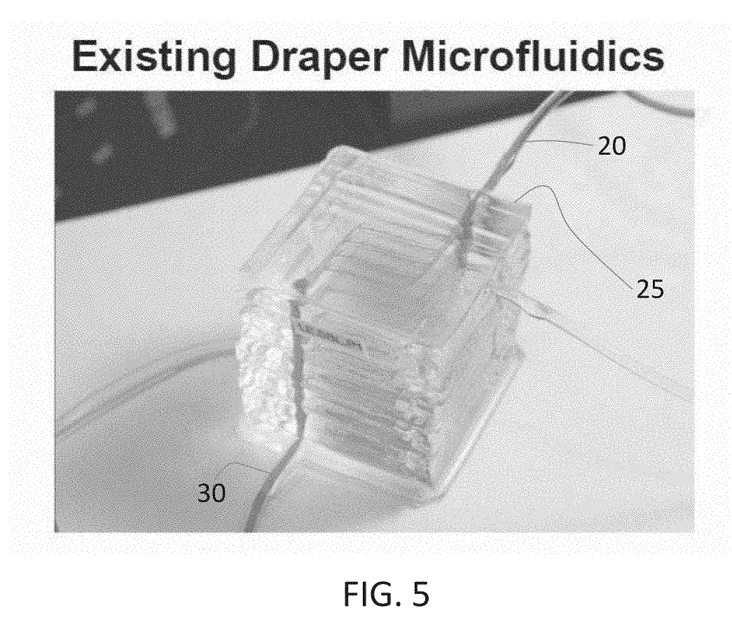

It is important to note that proteins that resonate in the lower frequency ranges (e.g., low GHz to kHz) would be more desirable targets, as the EM waves with these frequencies would propagate better through a human body with less off-resonance energy absorption caused by high frequency movement of all dipole molecules. In the THz band, where resonances for DNA and chromosomes are proven to exist, a frequency source could be used on blood in a microfluidic structure as shown in FIGS. 4 and 5 to, for example, destroy malignant cells in the bloodstream. In particular, the near-IR band, in which the first mode of resonance for most proteins exists with adequate tissue propagation at a higher power, is particularly suitable for the methods and devices described herein.

It is also reasonable to believe that an electromagnetic energy source with a much tighter bandwidth and more accurate tunable fixed frequency emissions than a common microwave can be developed to explore how electromagnetic resonances affect specific cells and proteins. Some of the proteins that could be explored specific to harmful human cell groups and viruses are described in the Example below, although in theory, any type of unwanted cell could be targeted.

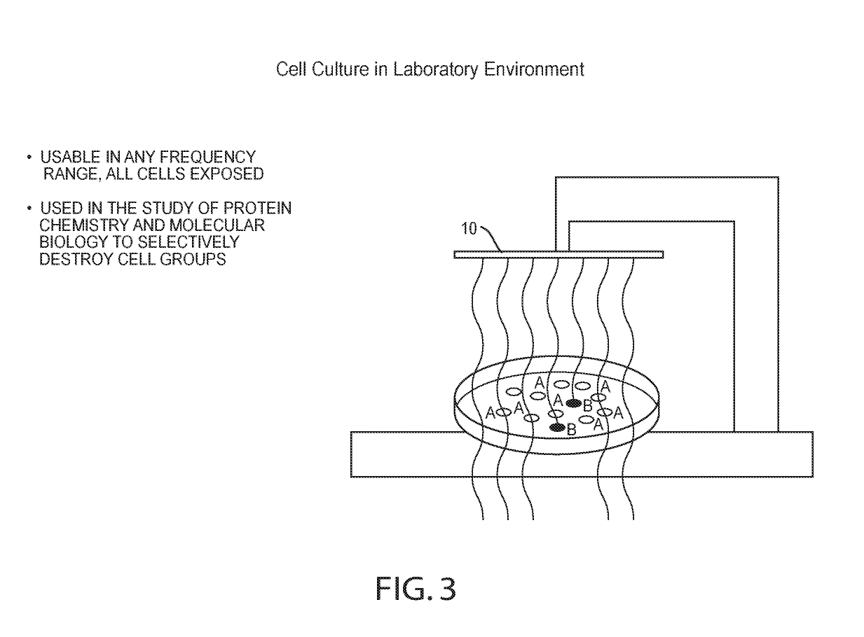

The methods described herein can be performed in vitro wherein the cells are maintained externally under cell culture conditions as known to those of skill in the art. Cell cultures typically contain a mixed population of cell types where only one population is desirable. Cell cultures can also be infected/contaminated with, for example, a virus or mycoplasma that can kill off the entire culture if left untreated. The methods described herein can be used to essentially completely eliminate, or substantially decrease, the contaminating cell types or cells containing infectious organism by targeting protein(s) and/or nucleic acid(s) that are specific for the contaminating cell types or organisms as shown in FIG. 3. In this figure the in vitro container dish contains two types of cells A (desired) and two undesired cells B. Both A and B are exposed to terahertz radiation from source 10. Only one cell type B will selectively be destroyed as it is the one that absorbs selected terahertz radiation, indicated by termination of wavy line indicating absorption of terahertz photons. The radiation safely passes through desired cell type A as indicated by the wavy lines passing through (not terminating) the A cells.

FIG. 4 is an embodiment of an ex vivo clinical use of higher frequency electromagnetic radiation where DNA and chromosome resonances are known to exist but the radiation cannot penetrate the body tissue of humans. The figure is an illustration of possible ex vivo treatment of cancer of the blood. The clinical steps involved are transporting the blood from the subject through 20 to a holding device 25 and exposing the blood to the resonant energy source 10 to kill most (if not all) of the cancerous cells. Following the destruction of the cancer cells, the blood can be pumped back into the patient through 30. In the figure the B cells are destroyed as indicated by absorption of photons, i.e., by termination of wavy lines from the electromagnetic source 10 at the B cells. The entire process must be carried out in a sterile environment. Input 20 and output 30 and the container 25 must be sterile. Optionally before being passed through 30 back to the body, blood can be filtered to eliminate dead cells. As shown in FIG. 5, the holding container can be a microfluidic device 25.

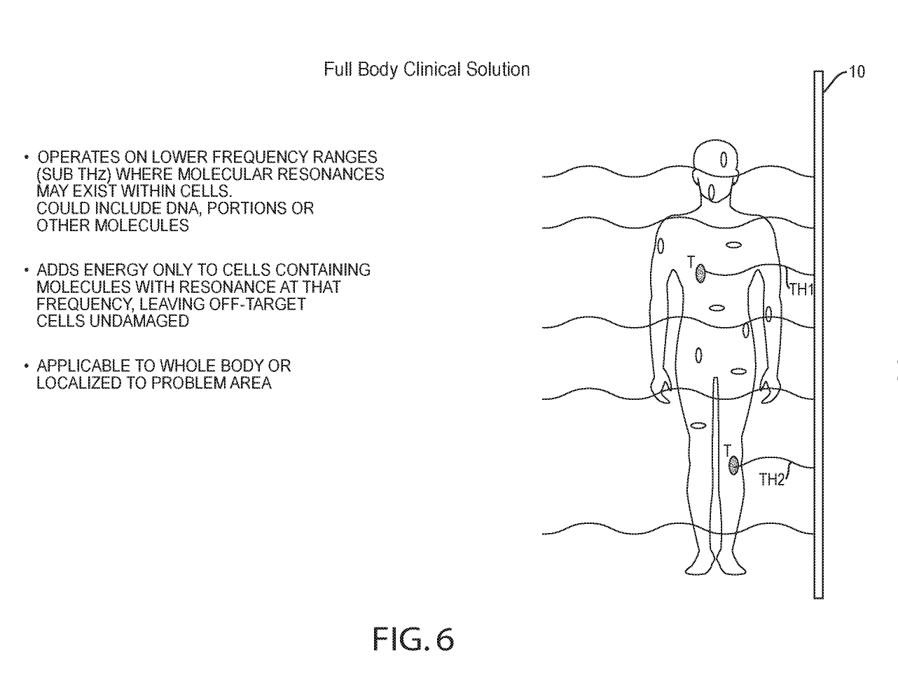

FIG. 6 is an embodiment of a full body system. The scanning system would operate on the whole body using lower frequencies where molecular resonances may exist within cells which could include DNA and portions of other molecules. This adds energy only to cells containing molecules with resonance at the frequency, leaving off-target cells undamaged. Such a system could be applicable to whole body or to a specific problem area. In the figure the electromagnetic source 10 aims radiation at whole body. Part of the radiation is absorbed by target cells T shown as slightly larger bright spots; the absorption is indicated schematically by wavy lines TH1 and TH2 which denote absorbed photons as these lines terminate inside the body at T. This method can be used both to diagnose (via imaging) target cells and to, possibly, destroy them.

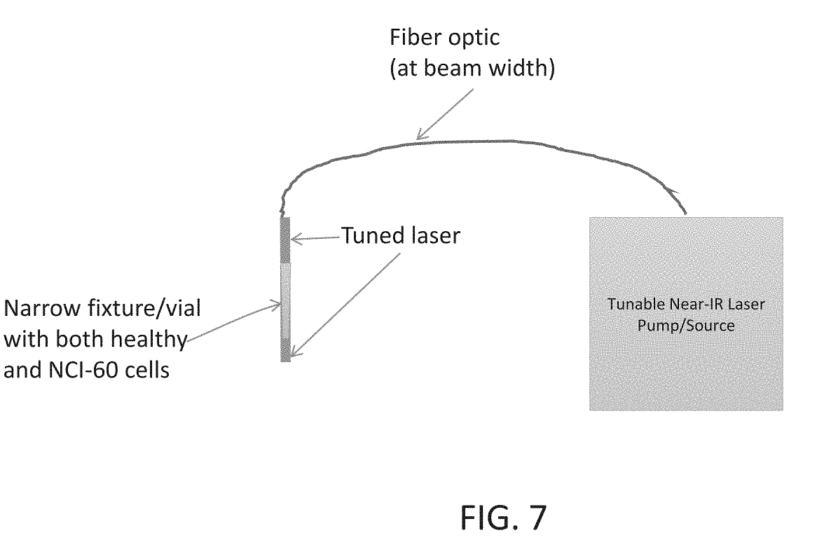

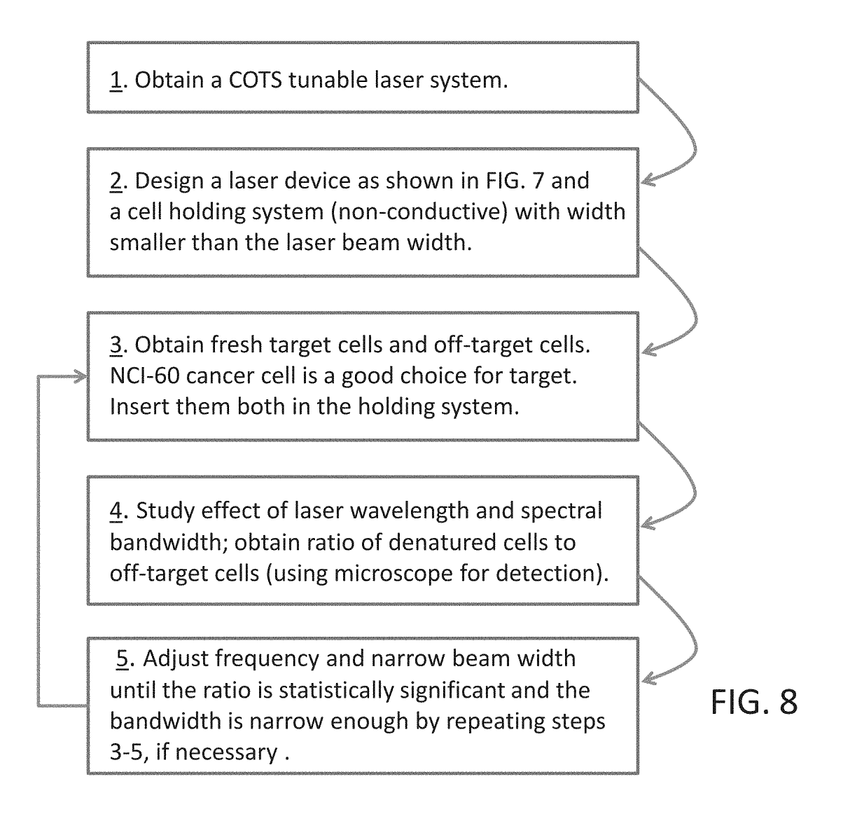

FIG. 7 is an embodiment at the laboratory deployment level of the concepts outlined in the invention. The figure shows tunable laser pump whose output is guided by a fiber optic guide to be output as radiation from the tuned laser. The laser output is then passed through a micro tube (holding device) containing cells to be treated. The tube can be a sterile non-conducting holding cell. This system is used in FIG. 8 to determine laser parameters, i.e., resonant frequencies and bandwidths. This system can be used during treatment to specifically target a biopsy or sample of target tissue.

More specifically, FIG. 8 lists a series of steps to determine laser resonant frequencies and bandwidths necessary for the use of laser system to denature undesirable cells. The process begins with acquiring a COTS (Commercial Off The Shelf) laser system in step 1 and creating a tunable laser system and a holding device in step 2. The holding device must be non-conductive and narrower than the laser beam width. In step 3 known quantities of both target and off-target cells are inserted into the holding device. In step 4, ratios of dead and off-target cells are computed after terahertz radiation. The cells can be evaluated by any number of assays know to those of skill in the art for determining the characteristics of living cells. For example, living and dead cells can be counted using a light or fluorescent microscope after an a time sufficient to allow the desired effect of the irradiation to occur, e.g., 1 minute, 1 hour, 1 day or 1 week. In practice, this identification, counting and iteration could be performed by a computer imaging and recognition system. Steps 3 and 4 are iterated (in step 5) until the ratio is statistically significant and bandwidth is as narrow as possible. The length of irradiation by the laser can also be determined this way.

Tunable near-IR laser components and systems suitable for the described methods are available from, for example NKT Photonics (e.g., the Fianium WhiteLase supercontinuum laser) or Thor Labs (tunable lasers). These components and systems can be further customized for the methods described herein.

The invention disclosed here has a number of applications, notably in the areas of research and clinical research as well as therapeutic applications as previously described.

Research applications: in microbiology and cell research, selectively destroying cells using electromagnetic resonance would create a currently non-existent capability of instantly destroying a specific cell type in a culture, saving time and money in some biological procedures. It would also allow for microbiologists to gain a deeper understanding of the functions of each protein type. For example, if one could excite a specific protein and denaturize it (perhaps using a shorter duration of energy to denaturize the protein structure while not harming the cell), one can then observe the effect that eliminating this protein has on the cell. This would have an academic impact, as well as aid in the creation of new medications and drugs.

Clinical applications: Implementing the methods described herein to treat patients suffering from cancers and infections could have a major impact on the medical field, giving physicians another tool to fight these difficult medical conditions. Most target resonant frequencies would be able to transmit through the body, allowing a physician to instantly treat diseases rather than spend precious time waiting for chemical actors to propagate throughout the body.

It is reasonable to believe that an electromagnetic radiation source that can maintain a single fixed frequency within the required level of precision throughout the entire body can be developed. In any practical implementation of this system (complex human system and non-ideal frequency source), some energy will be transmitted into non-target cells through similar proteins resonating or non-resonant effects. Although this can reasonably be overcome through experimentation and hardware development as known to those of skill in the art, a reasonable first step would be implementing the system in circulating blood only, eliminating blood borne cancers and diseases with limited off-target effects. This would allow the use a broader range of frequencies (body penetration issue is eliminated), acting as an intelligent dialysis device.

### Example: Target Proteins/Systems in Cells

Below are a few examples of proteins or nucleic acids that can be targeted for destruction/elimination using the methods and systems described herein. These proteins, receptors and other molecules are merely representative examples and the methods described herein can be used for the destruction of any cell comprising one, or more, proteins, nucleic acids, molecules or structures that can be identified by a unique natural resonance energy state suitable for excitation by the energy sources described herein.

EGF Receptor: These are simple, easily resonated proteins on the membranes of every human cell which are used to recognize normal chemical growth signals in the body. A cancer cell becomes malignant when these receptors are overexpressed, meaning that they exist in an order-of-magnitude higher than normal quantity which allows the cell to multiply uncontrollably. Targeting this would affect all cells of that tissue type in the body, but would be an improvement on existing radiation therapy as it would have a much greater effect on malignant tumors.

MAP kinase pathway or PI3K/Akt/mTOR pathway: Cycle containing multiple proteins that are different in mutated cells causing the creation of tumors and could be specifically targeted or tailored to discrete cancer types.

Apoptosis Inhibitors such as Inhibitor of Apoptosis (IAP) protein or Bcl-2 Proteins. These are proteins that exist in cancer cells preventing cells from dying by the apoptotic pathway.

Matrix metalloproteinases (MMPs) function in the extracellular environment of cells and degrade both matrix and non-matrix proteins allowing cancerous cells to metastasize to other tissues.

### Target Viral Proteins:

All viruses are made up of proteins, specific to the type of virus. These proteins fall into two categories—the structural proteins which make up the exterior or “shell” of the virus and the nonstructural proteins which contribute to the virus functionally. These proteins are all unique to viruses, and thus provide reasonable unique targets to effect virus destruction.

### Target Bacterial Proteins:

Bacteria contain some identical proteins to humans, and some independent proteins found only in bacteria that could be reasonably targeted.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of selectively destroying a cell or population of cells using electromagnetic resonance by targeting one, or more, specific protein(s) and/or nucleic acid(s) within the cell or cell population, comprising the steps of:
a) identifying the electromagnetic resonance properties of the target protein and/or nucleic acid within the cells;
b) providing an external energy source capable of generating sufficient energy or excitation within the cells to exploit the electromagnetic resonance properties identified in step a) and increase energy/heat within the cell(s); and
c) exposing the cells to the energy source in a manner and for a time sufficient wherein exposure to the energy source results in increased energy/heat within the cell(s) and in the selective death of cells containing the targeted protein/and or nucleic acid.

2. The method of claim 1, wherein the exploitation of electromagnetic resonance properties within the cell increases the friction of intermolecular bonds of the target protein and/or nucleic acid resulting in increased heat within the cell.

3. The method of claim 1, wherein the exploitation of electromagnetic resonance properties within the cell increases the energy level within the target molecule and totally or partially denatures the targeted protein and/or nucleic acid.

4. The method of claim 1, wherein the one, or more targeted protein(s) and/or nucleic acid(s) are specific for cancer cells, bacterial cells or viruses.

5. The method of claim 1, wherein the method is in vitro and the population of cells is maintained in cell culture, wherein the population contains one, or more, contaminating cell types, and the targeted protein(s) and/or nucleic acid(s) are specific for the contaminating cell types.

6. The method of claim 1, wherein the cells are present in a subject and the subject is selected from the group consisting of: a mammal, bird or reptile.

7. The method of claim 6, wherein the mammal is a human.

8. A ex vivo method of selectively destroying a cell or population of cells in the blood of a subject using electromagnetic resonance by targeting one, or more, specific protein(s) and/or nucleic acid(s) within the cell or cell population, comprising the steps of:
a) identifying the electromagnetic resonance properties of the target protein and/or nucleic acid within the cells;
b) providing an external energy source capable of generating sufficient energy or excitation within the cells to exploit the electromagnetic resonance properties identified in step a) and increase energy/heat within the cell(s);
c) transporting the blood from the subject through a sterile device and exposing the blood to the energy source provided in step b) to treat the blood and increase energy/heat within the cell(s), wherein the increased energy/heat within the cell(s) results in the selective death of cells containing the targeted protein/and or nucleic acid; and
d) transporting the treated blood back into the subject.

9. The method of claim 8, wherein the treated blood is optionally transported through a filter device to eliminate the dead cells from the treated blood before transporting the blood back into the subject.

10. The method of claim 8, wherein the subject is selected from the group consisting of: a mammal, bird or reptile.

11. The method of claim 10, wherein the mammal is a human.

12. A method of selectively destroying cells or a population of cells in the blood, tissue or organ of a subject using electromagnetic resonance by targeting one, or more, specific protein(s) and/or nucleic acid(s) within the cells or population of cells, comprising the steps of:
a) identifying the electromagnetic resonance properties of the target protein and/or nucleic acid within the cells;
b) providing an external energy source capable of generating sufficient energy or excitation within the cells to exploit the electromagnetic resonance properties identified in step a); and
c) supporting and positioning the subject's body in proximity to the energy source of step b); and
d) exposing the subject to the energy source in a manner and for a time sufficient for the increase of energy/heat within the cell(s), wherein the increased energy/heat within the cell(s) results in the selective death of cells containing the targeted protein/and or nucleic acid.

13. The method of claim 12, wherein the subject is selected from the group consisting of: a mammal, bird or reptile.

14. The method of claim 13, wherein the mammal is a human.

15. A method of selectively destroying cells or a population of cells in the blood, tissue or organ of a subject using electromagnetic resonance by targeting one, or more, specific protein(s) and/or nucleic acid(s) within the cells or population of cells, comprising the steps of:
a) identifying the electromagnetic resonance properties of the target protein and/or nucleic acid within the cells;
b) providing an external energy source capable of generating sufficient energy or excitation within the cells to exploit the electromagnetic resonance properties identified in step a); and
c) supporting and positioning the subject's body such that one, or more blood vessels, or blood vessels of the tissue or organ, are in proximity to the excitation generated by the energy source of step b); and
d) exposing the subject to the energy source in a manner and for a time sufficient for the increase of energy heat in proximity to the blood vessels, wherein the increased energy/heat in proximity to the blood vessel results in the selective destruction of the cell(s) within the blood vessels.

16. A system suitable for carrying out the methods of claim 1, the system comprising a device providing an external energy source capable of generating sufficient energy within the cells or population of cells to exploit the electromagnetic resonance properties of cells exposed to the energy source.
